(19) United States
(12) Patent Application Publication
(10) Pub. No.: US 2015/0041682 A1
(43) Pub. Date: Feb. 12, 2015

(54) Systems and Methods for Monitoring Phenanthrene Equivalent Concentrations

(71) Applicant: Benchmark Instrumentation & Analytical Services Inc., Edmonton (CA)

(72) Inventors: Amy Cano, Clovis, CA (US); Justin Caplinger, Fresno, CA (US); Keith Moffatt, Fresno, CA (US); Jonathan Harrison, Clovis, CA (US); David Doting, Morgan Hill, CA (US); Dale Brost, Coarsegold, CA (US)

(21) Appl. No.: 14/455,790

(22) Filed: Aug. 8, 2014

Related U.S. Application Data
(60) Provisional application No. 61/864,534, filed on Aug. 10, 2013.
(60) Provisional application No. 62/013,429, filed on Jun. 17, 2014.

(51) Int. Cl. G01N 21/64; G01N 33/18

(52) U.S. Cl. CPC G01N 21/64; G01N 33/1826
USPC 250/459.1; 250/458.1; 250/461.1

(57) ABSTRACT

The present application is directed to systems methods and methods for monitoring phenanthrene equivalent concentrations in an aqueous stream. One or more sources of electromagnetic radiation at different wavelengths may be directed into a sample of the aqueous stream and the resulting fluorescence at various wavelengths is detected. The detected fluorescence is then used to determine the phenanthrene equivalent concentration.

### CROSS-REFERENCE TO RELATED APPLICATIONS

The present application claims priority to provisional U.S. Patent Application Ser. No. 62/013,429, filed on Jun. 17, 2014, titled “Systems and Methods for Detection and Measurement of Impurities in Water Under Varying Color and Turbidity Conditions,” and provisional U.S. Patent Application Ser. No. 61/864,534, filed on Aug. 10, 2013, titled “Systems and Methods for Monitoring Phenanthrene Equivalent Concentration,” which are hereby incorporated by reference in their entirety.

### FIELD OF THE TECHNOLOGY

Embodiments of the disclosure relate to systems and methods capable of monitoring phenanthrene equivalent concentrations. More particularly, the embodiments disclosed herein relate to systems and methods for monitoring phenanthrene equivalent concentrations contained within the content of exhaust gas cleaning systems.

### BACKGROUND OF THE DISCLOSURE

By international agreement (MARPOL—the International Convention for the Prevention of Pollution from Ships), certain maritime areas in the Earth's waters are strictly governed. MARPOL was designed to minimize pollution of the seas, including dumping, and oil and exhaust pollution in furtherance of its stated objective to preserve the maritime environment through the complete elimination of pollution by oil and other harmful substances, and the minimization of accidental discharge of such substances. These regulations often limit what pollutants a ship may discharge into the air above the water, as well as what pollutants may be discharged into the water itself.

According to MARPOL, the polycyclic aromatic hydrocarbon (PAH) content of exhaust gas cleaning systems (EGCS) must be monitored continuously by UV absorption or fluorescence (depending on the water flow rate of the EGCS). This monitoring can be expressed in terms of phenanthrene equivalents (PAHphe).

Although the original intent of the regulation was to monitor PAH, it is now accepted that the actual organic contaminant in EGCS effluent is unburned heavy fuel oil (HFO). The actual analytical problem, therefore, is to monitor HFO contamination, express the contaminant concentration in units of PAHphe and to compensate for turbidity interference. Also, although not addressed in the regulations, EGCS manufacturers have stated that the measurement should also compensate for interference from gas bubbles that are often present in the effluent water stream.

The regulations specify the use of either UV absorption or fluorescence and units of PAHphe; however, they provided no guidelines as to how this measurement is to be instrumentally accomplished. It would be advantageous, for a variety of economic and operational reasons, if one instrument were available that could monitor EGCS effluents by either measurement technology to cover the entire PAHphe range that could be encountered during operation of any type of EGCS units. Further, it would be beneficial for the instrument to be able to monitor concentrations at least twice the permitted discharge limit to allow operators to track the recovery from process upsets. For this reason, the ideal instrument would be able to make measurements by UV absorption from 0-4500 μg/L PAHphe and fluorescence from 0-1800 μg/L PAHphe.

### SUMMARY

According to various embodiments, a method for measuring polycyclic aromatic hydrocarbon concentration is provided. The method may comprise passing a sample of an aqueous stream through a monitoring area. A first electromagnetic radiation may be directed into the monitoring area. The first electromagnetic radiation may have a first wavelength. A second electromagnetic radiation may also be directed into the monitoring area. The second electromagnetic radiation may have a wavelength different than the first wavelength. At least one fluorescence may be monitored when each of the first and second electromagnetic radiation is directed into the monitoring area. A polycyclic aromatic hydrocarbon concentration may be determined using at least one of the monitored fluorescence and may be reported as a phenanthrene equivalent concentration.

In accordance with additional embodiments, a device for measuring polycyclic aromatic hydrocarbon concentration is provided. The device may comprise a monitoring area, and a first and second source of electromagnetic radiation. Each of the first and second sources of electromagnetic radiation may be directed into the monitoring area. The first electromagnetic radiation may have a first wavelength. The second electromagnetic radiation may have a second wavelength different than the first wavelength.

The device may further comprise at least one sensor to detect at least one fluorescence. An analysis module may determine polycyclic aromatic hydrocarbon concentration using at least one of the detected fluorescence, and may be reported as a phenanthrene equivalent concentration.

In accordance with further embodiments, a method for measuring polycyclic aromatic hydrocarbon concentration is provided. The method may comprise obtaining a sample of an aqueous stream. A first electromagnetic radiation having a first wavelength may be directed into the sample, and a fluorescence from the sample at a second wavelength may be measured. The second wavelength may be greater than the first wavelength. A second electromagnetic radiation having a third wavelength may be directed into the sample, and a fluorescence from the sample at a fourth wavelength may be measured. The third wavelength may be greater than the first wavelength and different than the second wavelength. The fourth wavelength may be greater than the third wavelength. A polycyclic aromatic hydrocarbon concentration may be determined using the measured fluorescence at the second and fourth wavelengths, and may be reported as a phenanthrene equivalent concentration.

In accordance with yet additional embodiments, a non-transitory computer-readable media having computer-executable instructions for performing a method for measuring polycyclic aromatic hydrocarbon concentration. The instructions may comprise directing electromagnetic radiation having a first wavelength into a sample of an aqueous stream, then measuring at a second wavelength a fluorescence from the sample. The second wavelength may be greater than the first wavelength. The instructions may then comprise directing electromagnetic radiation having a third wavelength into the sample, then measuring at a fourth wavelength a fluorescence from the sample. The third wavelength may be greater than the first wavelength and different than the first wavelength. The fourth wavelength may be greater than the third wavelength. The instructions may further comprise determining the polycyclic aromatic hydrocarbon concentration of the sample using the measured fluorescence at the second and fourth wavelengths, and reporting the concentration as a phenanthrene equivalent concentration.

### BRIEF DESCRIPTION OF THE DRAWINGS

The accompanying drawings, where like reference numerals refer to identical or functionally similar elements throughout the separate views, together with the detailed description below, are incorporated in and form part of the specification, and serve to further illustrate embodiments of concepts that include the claimed disclosure, and explain various principles and advantages of those embodiments.

The methods and systems disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1 is a block diagram of an optical measurement system according to various embodiments.

FIG. 2 is a simplified block diagram of a 2-lamp, 2-detector optical measurement system according to various embodiments.

FIG. 3 is a side view of a lamp and detector arrangement about a flow cell according to various embodiments.

FIG. 4 is a top view of a lamp and detector arrangement about a flow cell according to various embodiments.

FIG. 5 is a side view of a lamp and detector arrangement about a flow cell according to various embodiments.

FIG. 6 is a top view of a lamp and detector arrangement about a flow cell according to various embodiments.

FIG. 7 is an exemplary flow chart of a method for measuring polycyclic aromatic hydrocarbon concentration of an aqueous stream according to various embodiments.

FIG. 8 is an exemplary flow chart of a method for measuring polycyclic aromatic hydrocarbon concentration of an aqueous stream according to various embodiments.

FIG. 9 is a block diagram of an exemplary computing device that may be used to implement various embodiments.

### DETAILED DESCRIPTION

The present application is directed to methods and devices for measuring polycyclic aromatic hydrocarbon concentration. The method may comprise passing a sample of an aqueous stream through a monitoring area. A first electromagnetic radiation may be directed into the monitoring area. The first electromagnetic radiation may have a first wavelength. A second electromagnetic radiation may also be directed into the monitoring area. The second electromagnetic radiation may have a wavelength different than the first wavelength. At least one fluorescence may be monitored when each of the first and second electromagnetic radiation is directed into the monitoring area. A polycyclic aromatic hydrocarbon concentration may be determined using at least one of the monitored fluorescence and may be reported as a phenanthrene equivalent concentration.

Various embodiments may comprise an optical measurement system that may be capable of performing fluorescence, absorption, reflectance and light scattering measurements. The measurements may be made individually to perform simple photometric measurements, or in any combination for complex monitoring tasks with multiple analytes, multiple samples, or where interference compensation is required.

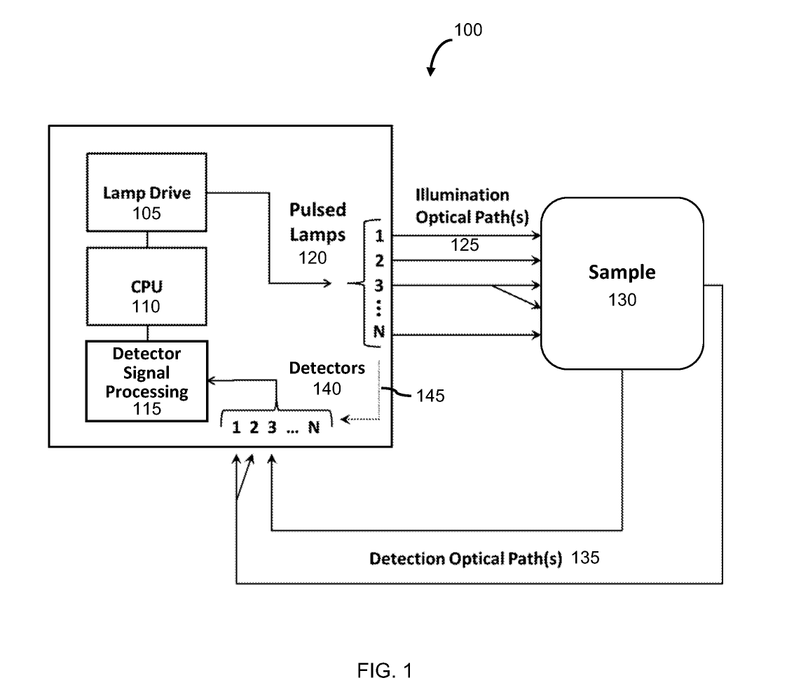

FIG. 1 illustrates a schematic diagram of an optical measurement system 100 according to various embodiments. The optical measurement system 100 may comprise a central processor unit (CPU) 110 controlling operation of a lamp drive unit 105 and a detector signal processing unit 115. The lamp drive unit 105 may drive one or more pulsed lamps 120. As each pulsed lamp 120 is operated, electromagnetic radiation (such as, but not limited to gamma rays, x-rays, ultraviolet light, visible light, near-infrared light, infrared light, microwaves, and radio waves) is emitted along one or more illumination optical paths 125 such that the electromagnetic radiation is directed into a sample 130. Electromagnetic radiation that passes through the sample 130 or is emitted by the sample 130 may travel along one or more detection optical paths 135 to one or more electromagnetic radiation detectors or sensors 140. Signals from the electromagnetic radiation detectors 140 may be directed to a detector signal processing unit 115, which may communicate with the CPU 110. Additionally, the CPU 110 and the detector signal processing unit 115 may contain memory for storing, for example, input settings and detector 140 readings.

The sample 130 is intended to represent any form of matter that can be photometrically or spectroscopically analyzed. The sample 130 may be contained in any type of chamber that is appropriate for the sample type and the desired analysis. This includes, but is not limited to, falling stream samplers, closed flow cells, gas samplers, cuvettes, and reflectance cups. The electromagnetic radiation may be directed to and from the sample 130 through open air, light guides, optical fibers, lenses, windows, and the like, and may be collected by any suitable detector 140 after interaction with the sample 130. Any type of fiber optic probe or other optical path 125, 135 may be used to carry the electromagnetic radiation from the lamps 120 to the sample 130 and from the sample 130 to the detectors 140.

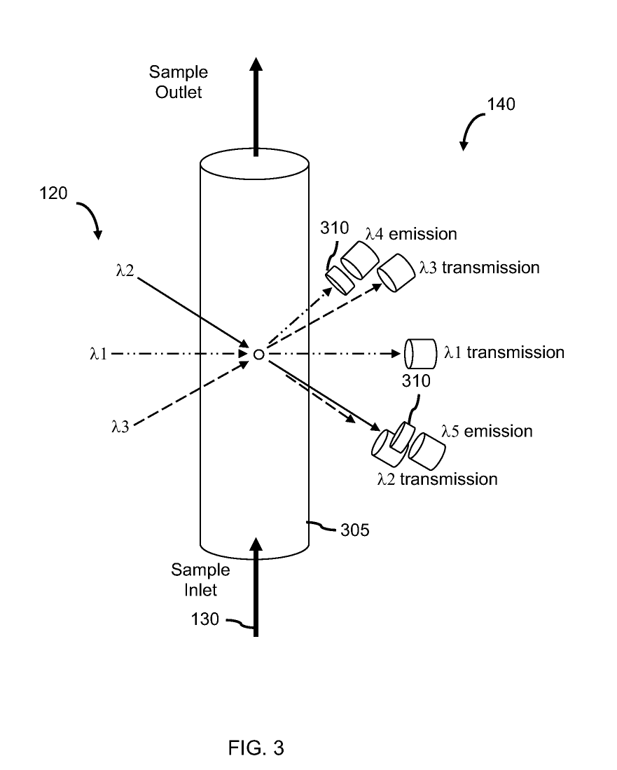

The lamps 120 may be any type of pulsed light source known in the art, such as xenon flash lamps, LEDs, lasers, and the like. The CPU 110 may communicate with the lamp drive unit 105 to pulse each lamp 120 in sequence such that only one lamp 120 is on at a time. The CPU 110 may set a pulse rate according to user input. All lamps 120 may be pulsed at the same rate, or each lamp 120 may be pulsed at different rates. The CPU 110 may independently control the optical power emitted by each lamp 120 by individually controlling the electrical power applied to the lamp 120. With xenon flash lamps 120, the optical power may be controlled by varying a discharge voltage. With LEDs lamps 120 and laser lamps 120, the optical power may be controlled by varying a drive current. The CPU 110 may automatically adjust the output power of each lamp 120 once each second (or another period of time as required by the specific analysis being performed and type of sample 130). The output power of each lamp 120 may be directed to the sample 130 through any type of optical path including air, light conduits, optical fibers, optical windows, and the like. Optical filters (not shown in FIG. 1, but illustrated in FIGS. 3 through 6) can be placed between the lamp 120 and the sample 130 to illuminate the sample 130 with a restricted wavelength range. An angle of incidence may be selected as appropriate for the desired measurement. The bifurcated illumination optical path 125 shown emerging from the third lamp 120 indicates that one lamp 120 could be used to illuminate the sample from multiple directions simultaneously.

The optical power of each lamp 120 may be monitored by a photodiode reference detector 145 represented by the dotted line between the lamps 120 and the detectors 140 in FIG. 1. An optical filter (not shown) may be placed between the lamp and the reference photodiode if it is desirable to restrict the bandpass of light monitored by the reference detector 145. Alternatively, photodiodes with specific wavelength sensitivities can be used. The CPU 110 may use the signal of each detector 140 and a PID control algorithm to maintain the optical power of each lamp 120 at a constant level.

Analytical detectors 140 for electromagnetic radiation pulses returning from the sample 130 may be any type known in the art such as photodiodes and photomultipliers that have the sensitivity and response time required to detect electromagnetic radiation pulses in the microsecond-millisecond range Optical filters may be placed between the sample 130 and the detectors 140 to illuminate the detectors 140 with a restricted wavelength range. In various embodiments, up to three detectors 140 can be associated with each lamp 120, including one reference photodiode 145 for monitoring the output power of the lamp 120 and two separate detectors 140 of the same or different types to monitor the electromagnetic radiation returning from the sample 130. The CPU 110 and detector signal processing unit 115 control the sensitivity of each type of detector 140 in a manner that is appropriate for the particular detector 140. If a photomultiplier is used, the magnitude of its output signal may be controlled by varying the high-voltage applied to its dynode chain. If a photodiode is used, the magnitude of its output may be controlled by varying the gain of an amplifier circuit. In either case, the signal is then integrated over the period of the electromagnetic radiation pulse, converted to a digital value and stored in memory for further processing. Additional sensitivity control may be achieved by varying the integration time of the detector signal (which, in turn, sets the duration of the lamp pulse). With the exception of photomultiplier high-voltage, which typically requires a 10 second settling time, parameters controlling the magnitude of detector response may be modified on a pulse-by-pulse basis. According to various embodiments, the gain and integration time adjustments described above may be carried out manually, may be carried out by software or firmware within the system 100, or may not be required depending on system 100 configuration.

Operational Overview—Optical Measurement System

The optical measurement system 100 may operate by causing the lamps 120 to emit pulses of electromagnetic radiation in rapid sequence. The pulses are used to stimulate a photometric response from the sample 130. Electromagnetic radiation pulses emerging from the sample 130, which correspond in time to the pulse emitted by the lamp 120, may be incident upon selected detectors 140, which convert the optical power to a proportional electrical signal. The signal is then amplified in a manner appropriate for the type of detector 140, integrated over the period of the electromagnetic radiation pulse, converted to a digital value and stored in memory for further computational processing.

Measurements may be performed one or more times per second, at a user-specified repetition rate. Lamps 120 may be pulsed in a predetermined sequence. One set of pulses from all active lamps 120 is termed a “pulse sequence.” Each pulse of each lamp 120 may result in a recorded measurement. At the end of each second (or other pulse period), the individual measurements may be averaged and reported to the user. The measurement rate may be set by the user from 1 to several hundred measurements per second. The maximum rate depends upon the number of lamps 120 and the period (integration time) of each pulse.

As mentioned previously, the lamp 120 output power may be held constant by PID control. The PID control is supplemented by a computational method described below. The sensitivity of drift-sensitive detectors 140, such as photomultiplier tubes, may be stabilized by a computational method, also described below, with the aid of a reference LED and a reference photodiode installed in the detector housing.

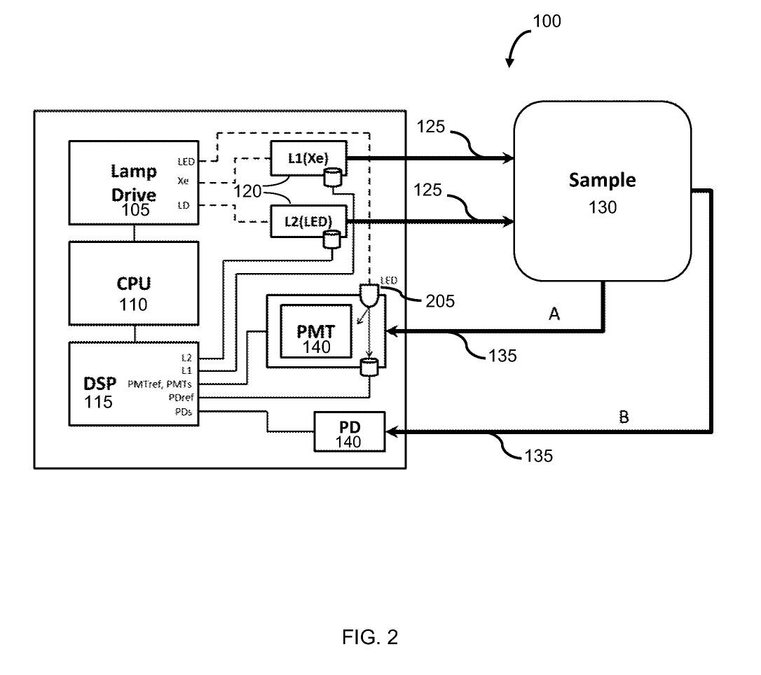

A more detailed diagram of the optical measurement system 100 according to various embodiments is shown in FIG. 2. The diagram represents a simplified embodiment of the system 100 that includes only two lamps 120; lamp 1 (L1) is a xenon flash lamp (Xe) and lamp 2 (L2) is an LED. Two detectors 140 are installed; a photomultiplier detector (PMT) and a photodiode detector (PD).

The lamps 120 (L1 and L2) are shown connected to the lamp drive unit 105 by dashed lines. The detector 140 connections are shown as thin solid lines. The electromagnetic radiation beams that travel to (125) and from (135) the sample 130 are shown as thick arrows. In addition to the xenon lamp (L1) and the LED (L2), which provide the electromagnetic radiation pulses that interact with the sample 130, a second LED lamp 205 may be located inside the PMT housing that provides reference electromagnetic radiation pulses for PMT drift compensation. The pulses may be simultaneously measured by both the PMT and a reference photodiode. In this example, the optical measurement system 100 is configured to measure the fluorescence and transmission of the sample 130. The fluorescence is excited by the xenon flash lamp L1, and the fluorescent emissions are measured by the photomultiplier (PMT) 140. The transmission of the electromagnetic radiation emitted by the LED L2 is detected by the photodiode (PD) 140.

The measurements of the various detectors 140 are shown in the detector signal processing unit 115 of FIG. 2. L1 may be proportional to the output power of the xenon flash lamp L1. L2 may be proportional to the output power of the LED L2. PMTs may be proportional to the optical power returning from the sample 130 along optical path A (135). PDs may be proportional to the optical power returning from the sample along optical path B (135). PMTref may be the response of the PMT 140 when the reference LED 205 is illuminated, and may be proportional to the optical power emitted by the reference LED 205.

According to various embodiments, the following events may occur during each pulse sequence. The current signals for all detectors 140 when the lamps 120 are dark (i.e., the lamps 120 are off) are measured and stored as a running sum. The lamps 120 are then pulsed in sequence. During each pulse, the signals from all of the illuminated detectors 140 are measured and stored as a running sum. These steps may then be repeated for a user-defined number of pulse sequences per second.

After each second, the average dark current for each detector 140 is computed by dividing the running sums by the user-defined number of pulse sequences per second. The average response for each illuminated detector 140 is computed by dividing the running sum by the user-defined number of pulse sequences per second. An amplifier offset is measured and subtracted from each averaged detector 140 response and dark current. The net detector 140 response is computed by subtracting the offset-adjusted dark current from the offset-corrected detector 140 response. The net detector 140 response for each of the lamp 120 reference photodiodes 140 is used to adjust the output power of each lamp 120 to the last value set by the user. The adjustment is determined by a PID algorithm that is pre-tuned for each lamp 120. The adjustment, if required, is made for the LED L2 by changing the LED L2 drive current. If an adjustment is required for the xenon flash lamp L1, it is made by changing the lamp discharge voltage. The reference LED 205 inside the PMT 140 housing is also automatically adjusted. Drift-compensated response parameter R1 is computed. Since the PMT in this example is a drift-sensitive device, R1 is computed by Equation 1:

$$R 1 = ( \frac{PMTs , adj}{L 1 adj} ) ( \frac{PDref , adj}{PMTref , adj} ) \qquad Eqn . 1$$

where,

PMTs,adj is the dark current and offset adjusted value of PMTs;

L1adj is the dark current and offset adjusted value of L1;

PDref,adj is the dark current and offset adjusted value of PDref; and

PMTref,adj is the dark current and offset adjusted value of PMTref.

Drift-compensated response parameter R2 is then computed. Since PD 140 is a stable detector 140 that is not affected significantly by temperature and aging, R2 is computed by Equation 2:

$$R 2 = ( \frac{PDadj}{L 1 adj} ) \qquad Eqn . 2$$

where,

PDadj is the dark current and offset adjusted value of PDs; and

L1adj is the dark current and offset adjusted value of L1.

A pseudo running average for each dark current and offset adjusted detector 140 response is computed over a time period specified by the use, and data are saved in memory. In various embodiments, data may be transferred to user interface electronics, such as a keypad, display, or PC, via an electronic or wireless connection. The transferred data may comprise two temperature measurements from two separate transducers. These measurements may be made a plurality of times per second and averaged over each one second period before the values are transferred to the user interface. These steps may be repeated as necessary.

Spectrometer Detectors

In various embodiments, the optical measurement system 100 may be designed to control and read the output of two (or more) spectrometers, such as CCD detectors 140. A typical device is of the type supplied by Ocean Optics Inc., Dunedin, Fla., USA. Many other suppliers of such devices exist and the optical measurement system 100 described herein is generally compatible with any of these devices.

Spectrometers measure optical power over a range of wavelengths simultaneously. Electromagnetic radiation enters an aperture and, after interaction with a diffraction grating, is wavelength-dispersed across the surface of the CCD detector 140. Each pixel in the detector 140 is illuminated by a different wavelength. The optical power is converted to a charge that is accumulated in the pixel's electron well during its exposure to electromagnetic radiation. The exposure time may be determined by the user and may correspond to an interval that produces enough charge for reliable measurements, but less than the saturation level of the electron well. At the end of this interval, the CPU 110 issues a read instruction to the CCD detector's control electronics and the data (digital values) is clocked out, on a pixel-by-pixel basis, and stored in memory for further processing.

The electromagnetic radiation is collected for a period of time (integration time) specified by the user. The integration time is selected by the user to give the sensitivity and range required for the measurement. For relatively high optical power levels, such as those encountered in transmission measurements, the integration time can be as short as the duration of a single electromagnetic radiation pulse. Relatively weak optical power levels, such as those encountered when measuring fluorescence, require longer exposure to light to accumulate enough charge to produce reliable signals. This is accomplished by allowing the CCD detector 140 to accumulate the charge produced from multiple electromagnetic radiation pulses before the readings are clocked out to the system 100 and reported to the user interface. Additional sensitivity and range control can be achieved by controlling the optical power output of the lamp 120 as well as the width of the pulse as described above for discrete detectors 140.

Automatic Setup

As described above, the user has detailed control over each lamp 120 and detector 140 installed in the system. These controls provide the flexibility to configure the system 100 for complex measurements involving several electromagnetic radiation sources 120 and detectors 140. Experienced users can easily set the system 100 up for complex measurements, but the task may be difficult for inexperienced users.

An automatic setup feature was developed to assist inexperienced users with the task of configuring the system 100 with the best combination of lamp 120 power and detector 140 sensitivity. With a sample 130 running through the system 100, the user estimates the percent full scale and enters the value. The automatic setup algorithm then sequentially determines the best settings for all electromagnetic radiation sources 120 and detectors 140. The results are then displayed for review by the user, who can then make any manual adjustment required.

Certain embodiments of the system 100 may comprise four lamps 120 and eight detectors 140. Multiple detectors 140 may be used for each lamp 120 used to perform the measurement. It can be a cumbersome process to set the correct levels of lamp 120 intensities and detector 140 gains to keep each measurement on-scale. Various embodiments of an automatic setup feature may reduce this process to three steps. First the user injects de-ionized (DI) water into a sample 130 chamber and sends a command to calibrate for DI water. Second, the user injects a known concentration of phenanthrene into the sample 130 chamber and sends a command to calibrate for phenanthrene. Lastly, the user injects a known concentration of HFO into the sample 130 chamber and sends a command to calibrate for HFO. At each step, the software takes repeated measurements and automatically adjust the levels of the light intensities and gain settings of the detectors used. With the exception of the PMT 140, the adjustment values are independently set for each lamp source 120. At the end of each step, the software saves the values for future use and indicates that it has completed measurement setup and is ready for the next step.

Since there is more than one adjustment that can be made to set the measured values on-scale, the auto setup algorithm assigns a weighted “cost” value to each adjustment. The goal is to achieve the measurement setup with the lowest value. The settings are initially set at mid-scale, which are assigned a zero “cost” value, allowing for the greatest adjustment if a disturbance occurred (temperature, time, etc.). As the adjustment moves farther away from the mid-scale its value goes up, “costing” more for that adjustment. This cost may be independently set for each adjustment and does not have to be linear with distance away from mid-scale.

### Example

All values used in this example are hypothetical and are used here to illustrate the Auto setup algorithm. Given Detector 1 is a photodiode and Lamp 1 is a laser with threshold current of 30 mA and maximum current of 100 mA. It is desired to get a reading of 4 on a 0-5 scale from the Detector 1 signal in conjunction with Lamp 1. The adjustments for this measurement are shown in Table 1:

TABLE 1
Range of Adjustments and Costs

| | Range of | |
|---|---|---|
| Adjustment | Adjustment | Cost |
| TIME (μs) | 25 to 50,000 | 0, for Time < 500 |
| | | 0.33 (Time-500), for Time > 500 |
| GAIN | 1 to 255 | 0, for Gain = 64 to 128 |
| | | 64-Gain, for Gain < 64 |
| | | Gain -128, for Gain > 128 |
| CURRENT (mA) | 30 to 100 | 5*\|Current-65\| |

The algorithm first sets TIME=250, GAIN=96, and CURRENT=65. The algorithm then measures the signal from Detector 1 and executes the following routine:

If Detector 1<4
Proportionally increase TIME to 500
Cost=0
Else proportionally decrease TIME to 25
Cost=0
Re-measure Detector 1
If Detector 1=4
Done
Cost=0
Else if Detector 1<4, proportionally decrease GAIN to 64
Cost=0
Re-measure Detector 1
If Detector 1=4
Done
Cost=0
Else if Detector 1<4, adjust CURRENT, GAIN, TIME based on least
cost
Cost≠0
Re-measure Detector 1
If Detector 1=4
Done
Cost≠0

This example shows the iterative process used for one detector 140 exposed to one lamp 120. The actual auto setup would be measuring multiple detectors 140 from each lamp source 120 and making adjustments to all lamps 120 and detectors 140 every second. As mentioned previously, the gain and integration time adjustments described above may be carried out manually, may be carried out by software or firmware within the system 100, or may not be required depending on system 100 configuration.

Using the devices and methods described above, the following observations have been made with respect to the measurement of PAH content of EGCS:

Absorption of UV Light
1. Phenanthrene and HFO absorb electromagnetic radiation in the short-wavelength (short-λ) ultraviolet region of the electromagnetic spectrum. The absorbance for both substances is linear with concentration over the entire concentration range needed for monitoring EGCS units with flow rates <2.5 t/MWh (0-4500 μg/L PAHphe).
2. To minimize the challenge to EGCS manufacturers and ship owners, the absorption measurements should be made at the wavelength that gives the highest absorbance for a given concentration of phenanthrene and the lowest absorbance for a given concentration of HFO. This wavelength corresponds to the peak of the phenanthrene absorption spectrum.
3. The absorption of short-λ UV light is strongly affected by the presence of turbidity from suspended solids and from air bubbles. Compensation may be required for measurements to accurately indicate the PAHphe concentration of the effluent water.

Fluorescence
1. Phenanthrene emits strong fluorescence over a very narrow band of wavelengths in the short-λ ultraviolet portion of the electromagnetic spectrum.
2. HFO emits fluorescence over a very broad band of wavelengths from the short-λ ultraviolet to the near infrared.
3. To express HFO concentration in phenanthrene equivalents, the fluorometer must be able to detect both. In other words, the sensitivity and measurement ranges for both phenanthrene and HFO must be appropriate for the PAHphe range permitted for the EGCS water flow rate.
4. The PAHphe concentration of HFO is a function of the excitation and emission wavelength used in the analysis, and can change over a factor of 200 or more over the wavelength range where phenanthrene emits strong fluorescence.
5. To minimize the challenge to EGCS manufacturers and ship owners, the fluorometer should be operated at wavelengths that give the lowest PAHphe value for a given concentration of HFO. This corresponds to the very narrow wavelength range in the short-λ UV where phenanthrene emits its strongest fluorescence.
6. Although short-λ UV wavelengths are ideal for measuring the low concentrations permitted for high-rate EGCS units, fluorescence-based measurement of the high concentrations permitted for low-rate units (2.5-11.25 t/MWh) require longer wavelengths to achieve an adequate linear range. These longer wavelengths, however, cannot be used exclusively, because the instrument would not be sensitive to phenanthrene and therefore could not be calibrated in PAHphe units.
7. Air bubbles are common in EGCS effluents and their presence substantially increases fluorescence-based PAHphe readings. Compensation may be required for measurements to accurately indicate the PAHphe concentration of the effluent water.
8. Turbidity from soot particles is also common and acts in the opposite direction substantially decreasing reported PAHphe concentration.

In light of the above observations, various embodiments comprise an instrumental method that performs fluorescence measurements at two different sets of excitation (EX) and emission (EM) wavelengths. The first (short-λ) EX/EM pair corresponds to the wavelengths where phenanthrene emits maximum fluorescence. The second (long-λ) EX/EM pair corresponds to the wavelengths that yields the dynamic range required to monitor the highest expected HFO concentration. Various embodiments may also comprise methods that provide the signals required for removing interferences from absorption and fluorescence measurements resulting from gas bubbles and suspended solids. This method may comprise the use of a near-infrared (NIR) light source 120 and a detector 140 for detecting the NIR light transmitted by the sample 130 and a detector 140 to monitor the long-λ excitation light that is transmitted by the sample 130.

Various embodiments may comprise a calibration method that relates the short-λ fluorescence response to phenanthrene concentration (primary PAHPhe calibration) and that relates the long-λ fluorescence response to the short-λ calibration (secondary PAHPhe calibration). Additional embodiments may comprise methods for compensating absorption-based PAHphe measurements and fluorescence-based PAHphe measurements for interferences due to suspended solids and gas bubbles.

The methods described herein may be carried out using an optical measurement system 100 such as that described above in relation to FIGS. 1 and 2. The system 100 may comprise a flow cell to contain a water sample 130, constructed of a material capable of at least partially transmitting electromagnetic radiation at the wavelengths described below. The system may also comprise a source 120 of short-wavelength UV light (λ1) for measuring phenanthrene by ultraviolet absorption and/or for exciting phenanthrene fluorescence, a detector 140 for detecting the λ1 light that is transmitted by the sample, and a detector 140 for the detection of phenanthrene fluorescence at a UV wavelength (λ4) that is longer than the excitation wavelength λ1.

The excitation/emission wavelengths represented by λ1 and λ4, respectively, correspond to the peak of the phenanthrene emission spectrum. This may occur when λ1 is a wavelength bandpass from about 240 nm to about 260 nm and λ4 is a wavelength bandpass between about 350 nm and about 370 nm. The exact best selection of λ1 and λ4 can vary somewhat, depending on the output spectrum of the excitation light source 120, the wavelength-dependent transmission efficiency of the optical path 125, 135, the relative orientation of the excitation and emission light beams, the wavelength resolution (bandpass) of λ1 and λ4 and the spectral sensitivity characteristics of the detector 140. An angle between the excitation (λ1) and emission (λ4) light beams may range from about 80 degrees to about 110 degrees, but may vary between about 0 and about 180 degrees.

The system 100 may also comprise a source 120 of UV/VIS wavelength light (λ3), at a wavelength longer than λ1, for exciting HFO fluorescence, and a detector 140 for the detection of HFO fluorescence at a UV/VIS wavelength (λ5), that is longer than the excitation wavelength λ3. In various embodiments the λ3 wavelength bandpass ranges from about 390 nm to about 410 nm. The excitation/emission wavelengths represented by λ3 and λ5, respectively, are any wavelengths that give fluorescence response that varies with HFO concentration such that: a) fluorescence measured with λ3/λ5 is linear with HFO concentration (expressed in units of PAHphe) and above the level required for reliable analytical measurements of HFO concentration in an HFO concentration range where the fluorescence measured with λ1/λ4 is a linear function of HFO concentration; and b) fluorescence measured with λ3/λ5 is linear with HFO concentration up to the maximum concentration expected in EGCS effluent water (˜1800 μg/L PAHphe). An angle between the excitation (λ3) and emission (λ5) light beams may range from about 80 degrees to about 110 degrees, but can vary between about 0 and about 180 degrees.

Further embodiments of the system 100 may comprise a source 120 of near-infrared (NIR) light (wavelength λ2), selected so that measurement of the transmitted NIR light at an angle ranging from about 90 degrees to about 180 degrees from the angle of incidence can be used alone or in conjunction with the transmitted light at λ1 and/or λ3 to compensate the λ1 absorbance measurements and the λ1/λ4 and λ3/λ5 fluorescence measurements for interference due to gas bubbles and turbidity.

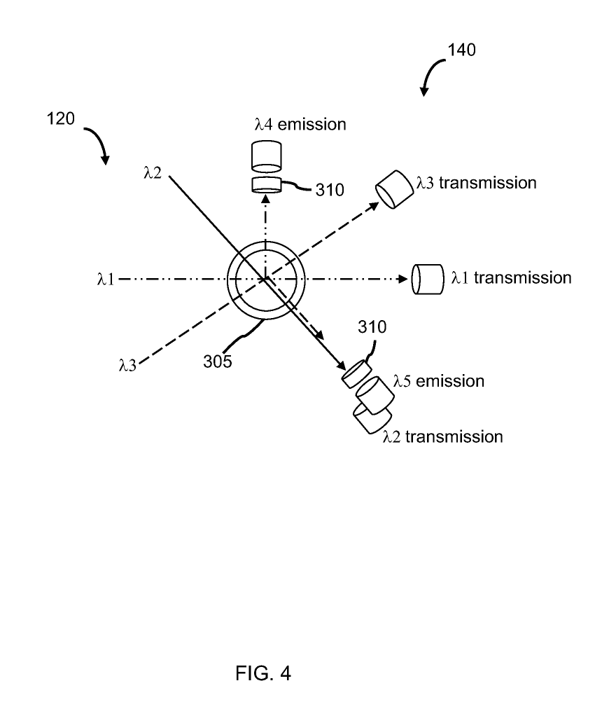

FIG. 3 schematically illustrates a side view and FIG. 4 schematically illustrates a top view of an arrangement of lamps 120 and detectors 140 around a flow cell 305 according to various embodiments. The lamps 120 and detectors 140 may be arranged in the same plane, or in two or more planes as required to physically accommodate the lamps 120 and detectors 140. It is also possible to place dichroic or long-pass optical filters 310 in front of a detector 140 to allow that detector 140 to detect more one light beam. It is also possible to use a wavelength-selective detector 140, such as a spectrometer, to detect multiple wavelengths simultaneously by coupling the wavelength-selective detector 140 to the light beams with light guides or optical fibers.

In FIGS. 3 and 4, two detectors 140 are shown on the λ2 optical axis. One detector 140 is unfiltered for detection of λ2 transmitted light. The other detector 140 is filtered with a λ5 bandpass filter 310 for detection of fluorescent emissions resulting from excitation with λ3.

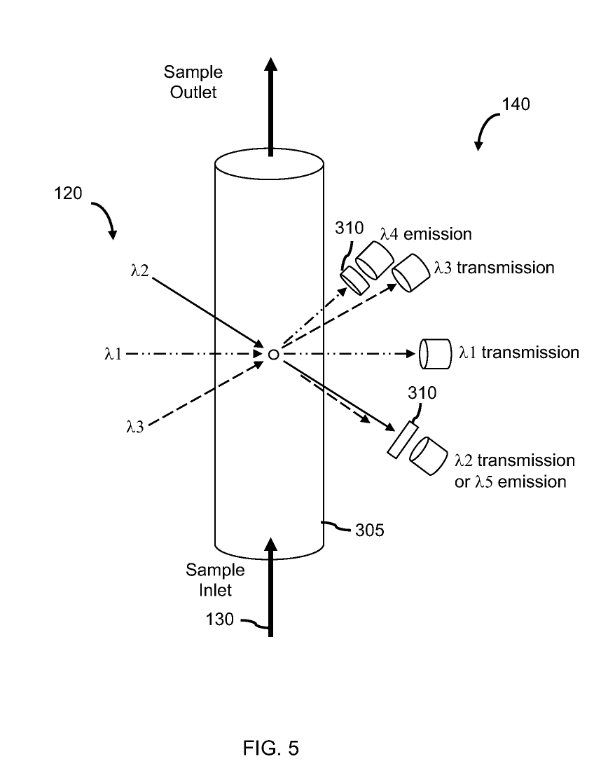

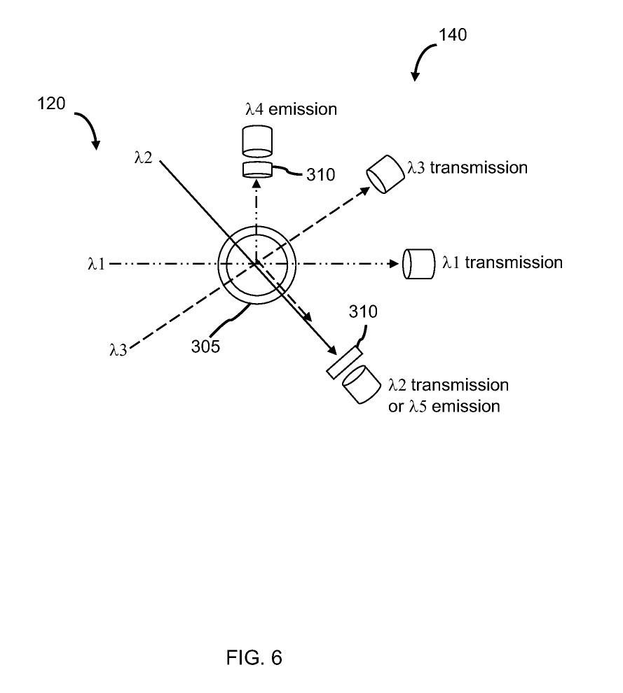

FIGS. 5 and 6 illustrate various embodiments of an alternate configuration using one detector 140 to detect light on the λ2 optical axis. In this configuration, a long pass filter 310 may be located in front of the detector 140 on the λ2 axis. The filter 310 transmits all wavelengths longer than its cut-on wavelength, passing both λ2 as well as the fluorescence excited by λ3 at a wavelength of λ5.

The detectors 140 may be positioned as shown in FIGS. 3 through 6 because of the unique design of the optical measurement system 100 described above. The system 100 operates by generating sequential pulses of electromagnetic radiation and integrating specific detector 140 signals over the period of each pulse. Only one light source 120 is illuminated at a time. Specific detectors 140 are associated in firmware with each source 120 pulse, so that only the signals that are appropriate at the time of each pulse are measured.

UV Absorbance Calibration

Since the absorbance of λ1 light is a linear function of phenanthrene and HFO concentration over the concentration range of interest for both, calibration is achieved by normal methods known to those familiar with photometric measurements. To conform to the concept of PHE equivalents, calibration must be performed by correlating the concentration of aqueous solution(s) of phenanthrene to the absorbance measured for the same solution(s).

For the measurement of high concentration of HFO and/or phenanthrene, UV absorbance is used. Based on preliminary test results, it appears that both HFO and phenanthrene absorb light in the short UV range; meaning that λ1 could be used. The data appeared to give linear response well beyond the required limit and with an LOQ below the linear range of the secondary fluorescence measurement.

Basis for “Cross-wavelength” Fluorescence Calibration

Due to wavelength-specific sensitivity and linear range characteristics, fluorescence calibration cannot be achieved over the entire concentration range of interest by simply correlating measured fluorescence at a single excitation/emission wavelength pair to the concentration of aqueous phenanthrene solutions. The “cross-wavelength” calibration method, described in detail below, is based on fluorescence measurements at two different excitation/emission wavelength pairs. The method makes it possible to calibrate fluorescence measurements in accordance with MARPOL regulations and to enable the fluorescence technique to cover the entire concentration range permitted for all types of EGCS units.

The method is fundamentally based on primary calibration with aqueous solutions of phenanthrene, with fluorescence measurements made at the peak of the phenanthrene fluorescence spectrum. These wavelengths are represented by λ1 (excitation) and λ4 (emission) in FIGS. 3 through 6. The primary calibration allows the instrument to convert fluorescence measured at λ1/λ4 directly to phenanthrene equivalents (PAHphe). Fluorescence at λ1/λ4 wavelengths are very sensitive and make it possible to reliably report PAHphe at the ultra-low levels required by high water-rate EGCS units. However, with a typical 10 mm fluorescence cell and a typical angle of about 90 degrees between the excitation and emission beams, fluorescence at λ1/λ4 is a linear function of concentration up to about 250 μg/L PAHphe. At higher concentrations, the instantaneous calibration slope (fluorescence per concentration unit) decreases progressively until it reaches zero, resulting in a practical measurement limit of approximately 1000 μg/L PAHphe. This concentration is far below the maximum concentration range required to monitor low water-rate EGCS units during upset conditions.

To solve the high-range problem, fluorescence is also measured at the longer wavelengths required to provide extended linear range for HFO. These wavelengths are represented by λ3 (excitation) and λ5 (emission) in FIGS. 3 through 6. However, fluorescence measured at λ3/λ5 is not sensitive to phenanthrene and therefore cannot be directly calibrated with aqueous solutions of phenanthrene. Instead, a method termed “cross-wavelength” calibration is used to achieve a secondary λ3/λ5 calibration that is referenced to the primary λ1/λ4 calibration. The cross-wavelength method makes it possible to express the fluorescence measured at λ3/λ5 in units of PAHphe even though phenanthrene itself cannot be detected at λ3/λ5. Low concentrations are determined by measuring fluorescence at λ1/λ4 and converting the measurement to PAHphe using the primary calibration slope. High concentrations are determined by measuring fluorescence at λ3/λ5 and converting the measurement to PAHphe using the secondary calibration slope. PAHphe concentrations determined at λ3/λ5 are equivalent to the value that would have been obtained at λ1/λ4, if the fluorescence at λ1/λ4 had remained linear over the entire concentration range.

The combination of measuring fluorescence at two excitation/emission wavelengths and the cross-wavelength calibration method allows the instrument to determine the PAHphe concentration of EGCS effluents over the entire range of interest for all EGCS types.

Cross-Wavelength Calibration Method

The method and example procedure presented herein are meant to describe the method to those generally familiar with photometric and spectroscopic measurements. Many variations are possible, especially with respect to solution preparation and the order of operations. For simplicity, the procedure described below assumes a linear relationship between fluorescence and concentration, which is attainable within suitable concentration limits with the proper selection of λ1/λ4 and λ3/λ5. However, it is also possible to use non-linear fluorescence versus concentration relationships with more complex calculations.

The term “measurement cell” as used herein refers to any type of optically transparent container 305 in which the water sample 130 resides or through which the water sample 130 passes, including fiber optic fluorescence probes. The instrument may be configured as described in FIGS. 3 and 4, or as described in FIGS. 5 and 6, or in an alternative manner as described herein.

In the mathematical procedure described below, the following terminology is used:

R_{blank,λ1/λ4} Measured fluorescence response for blank water at λ1/λ4

R_{blank,λ3/λ5} Measured fluorescence response for blank water at λ3/λ5

C_{PHE} Concentration of phenanthrene standard in μg/L

C_{HFO,High} Concentration of high concentration HFO standard in μg/L

C_{HFO,Low} Concentration of low concentration HFO standard in μg/L

R_{PHE,λ1/λ4} Measured fluorescence response for phenanthrene standard at λ1/λ4

R_{HFO,High,λ3/λ5} Measured fluorescence response for the high concentration HFO standard at λ3/λ5

R_{HFO,Low,λ1/λ4} Measured fluorescence response for the low concentration HFO standard at λ1/λ4

R_{S,λ1/λ4} Measured fluorescence response for the water sample at λ1/λ4

R_{S,λ3/λ5} Measured fluorescence response for the water sample at λ3/λ5

[PAHphe] The phenanthrene equivalent concentration of the sample in μg/L

Mathematical Definition of Cross-Wavelength Method

Compute the primary PAHphe calibration slope,

$${m}_{\frac{λ1}{λ4}} ,$$

using equation 3:

$${m}_{\frac{λ1}{λ4}} = \frac{{C}_{PHE}}{{R}_{PHE \frac{λ1}{λ4}} - {R}_{blank \frac{λ1}{λ4}}} \qquad Eqn . 3$$

Compute the phenanthrene equivalent concentration of a low concentration HFO standard, [PAHphe]_{HFO,Low}, using Equation 4:

$${[ PAHphe ]}_{HFO , Low} = {R}_{HFO , Low \frac{λ1}{λ4}} ( {m}_{\frac{λ1}{λ4}} ) \qquad Eqn . 4$$

Compute the phenanthrene equivalent concentration of a high concentration HFO standard, [PAHphe]_{HFO,High}, using Equation 5:

$${[ PAHphe ]}_{HFO , High} = \frac{{{C}_{HFO , High} [ PAHphe ]}_{HFP , Low}}{{C}_{HFO , Low}} \qquad Eqn . 5$$

Compute secondary calibration slope,

$${m}_{\frac{λ3}{λ5}} ,$$

using Equation 6:

$${m}_{\frac{λ3}{λ5}} = \frac{{[ PAHphe ]}_{HFO , High}}{{R}_{HFO , High \frac{λ3}{λ5}} - {R}_{HFO , Low \frac{λ3}{λ5}}} \qquad Eqn . 6$$

Example Procedure

Place a blank in the measurement cell 305. The term “blank” refers to water that is uncontaminated with phenanthrene or HFO. In the present context, the “blank” is the feed water entering the EGCS unit.

Measure and record the fluorescence responses of the blank at λ1/λ4, R_{blank,λ1/λ4} and at λ3/λ5, R_{blank,λ3/λ5}.

Prepare a phenanthrene standard solution by dissolving phenanthrene in blank water. The concentration of the phenanthrene standard solution, C_{PHE}, should be in the concentration range where λ1/λ4 fluorescence is linear with phenanthrene concentration.

Place the phenanthrene standard in the measurement cell 305. Measure and record the fluorescence response of the phenanthrene standard at λ1/λ4, R_{PHE,λ1/λ4}.

Prepare a stock HFO solution by dispersing HFO in isopropanol with the aid of an ultrasonic bath.

Dilute the stock HFO standard with blank water to a relatively low concentration, C_{HFO,Low}, known to yield fluorescence at λ1/λ4 that is linear with HFO concentration.

Place the low concentration HFO standard in the measurement cell 305. Measure and record the λ1/λ4 fluorescence response, R_{HFO,Low,λ1/λ4}.

Dilute the stock HFO standard with blank water to a relatively high concentration, C_{HFO,High}, where C_{HFO,High} is Much greater than C_{HFO,Low}, greater than the linear range for HFO measured at λ1/λ4, and known to be in the linear range for HFO measured at λ3/λ5.

Place the high concentration HFO standard in the measurement cell 305. Record the λ3/λ5 fluorescence response (R_{HFO,High,λ3/λ5}).

Analysis

While monitoring actual EGCS effluents, the system 100 continuously produces sample fluorescence measurements at λ1/λ4, R_{S,λ1/λ4}, and λ3/λ5, R_{S,λ3/λ5}. The system's firmware uses the relative magnitudes of sample R_{S,λ1/λ4} and R_{S,λ3/λ5} to automatically determine the best wavelength to use to report PAHphe. The logic is described below:

1. If R_{S,λ1/λ4}>R_{S,λ3/λ5}, use R_{S,λ1/λ4} for maximum sensitivity.

2. If R_{S,λ1/λ4}≈R_{S,λ3/λ5}, use R_{S,λ3/λ5}, because R_{S,λ1/λ4} may be a non-linear function of PAHphe concentration.

3. If R_{S,λ3/λ5}>R_{S,λ1/λ4}, use R_{S,λ3/λ5} for maximum linear range.

If the R_{S,λ1/λ4} measurement is selected, the PAHphe concentration, [PAHphe] is computed using Equation 7:

$$[ PAHphe ] = {R}_{s , \frac{λ1}{λ4}} ( {m}_{\frac{λ1}{λ4}} ) \qquad Eqn . 7$$

If the R_{S,λ3/λ5} measurement is selected, the PAHphe concentration is computed using Equation 8:

$$[ PAHphe ] = {R}_{s , \frac{λ3}{λ5}} ( {m}_{\frac{λ3}{λ5}} ) \qquad Eqn . 8$$

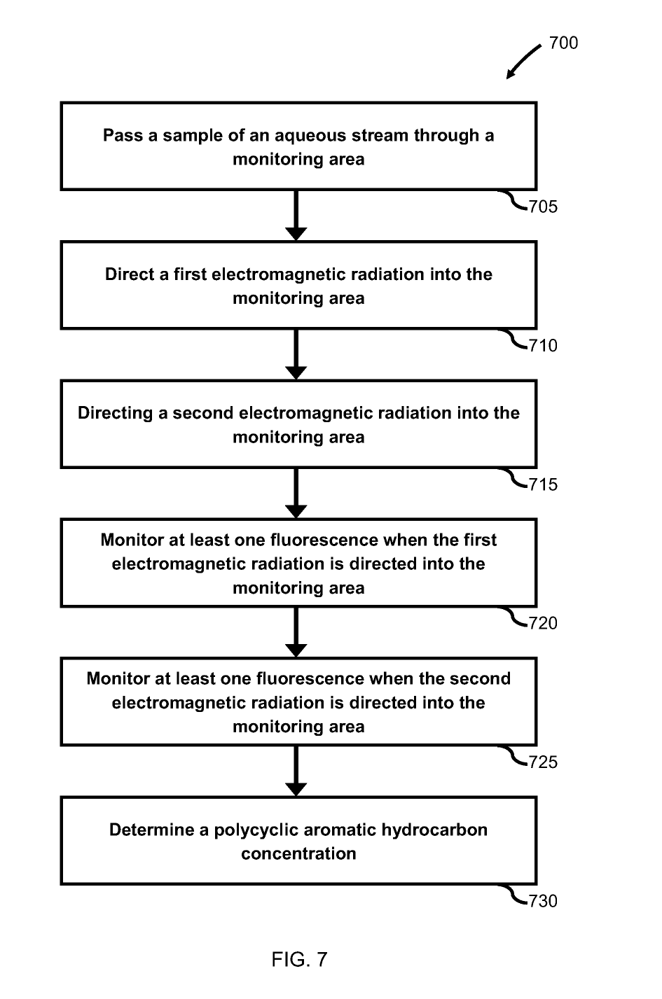

FIG. 7 illustrates a flow chart of an exemplary method 700 for measuring polycyclic aromatic hydrocarbon concentration of an aqueous stream according to the methods described herein. The method 700 may comprise a step 705 of passing a sample of an aqueous stream through a monitoring area, such as the flow cell 305. The monitoring area may have a plurality of lamps 120 (sources of electromagnetic radiation) and detectors 140 arrayed around the monitoring area. At step 710, a first electromagnetic radiation may be directed into the monitoring area. The first electromagnetic radiation may have a first wavelength. A second electromagnetic radiation having a second wavelength may be directed into the monitoring area at step 715.

The method 700 may further comprise a step 720 of monitoring at least one fluorescence when the first electromagnetic radiation is directed into the monitoring area. Similarly, the method 700 may comprise a step 725 of monitoring at least one fluorescence when the second electromagnetic radiation is directed into the monitoring area. Each fluorescence may be sensed by one or more detectors 140, such as detectors 140 in FIGS. 1 and 2. At step 730, a polycyclic aromatic hydrocarbon concentration may be determined and reported as a phenanthrene equivalent concentration using one or more of the monitored fluorescence.

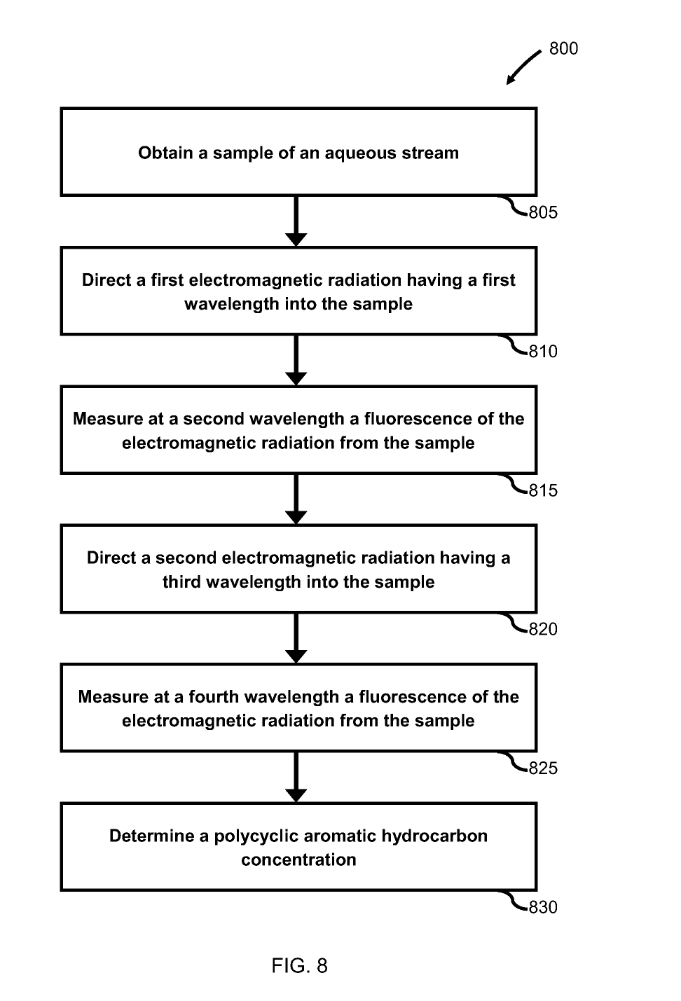

Turning now to FIG. 8, shown therein is an alternate exemplary method 800 for measuring polycyclic aromatic hydrocarbon concentration of an aqueous stream. The method 800 may comprise a step 805 for obtaining a sample of the aqueous stream. At step 810, a first electromagnetic radiation having a first wavelength may be directed into the sample, and then measuring at a second wavelength a fluorescence of the electromagnetic radiation from the sample at step 815. The second wavelength may be greater than the first wavelength. The method 800 may further comprise step 820 in which a second electromagnetic radiation having a third wavelength is directed into the sample. The third wavelength may be greater than the first wavelength and different than the second wavelength. At step 825, a fluorescence of the electromagnetic radiation may be measured from the sample at a fourth wavelength. The fourth wavelength may be greater than the third wavelength. The polycyclic aromatic hydrocarbon concentration of the sample may be determined at step 830. The concentration may be determined using the measured values for fluorescence at the second wavelength and fluorescence at the fourth wavelength. The concentration may be reported as a phenanthrene equivalent concentration.

Elimination of the Turbidity Interference

Turbidity can drastically affect fluorescence readings by blocking light. A turbidity compensation method may be used for both the primary (λ1) and secondary (λ3) fluorescence measurements. For the primary fluorescence measurement, a multivariate data analysis method may be used (multiple linear regression). This would require the use of software to store data in matrix form and compute a regression matrix that can be used to solve for unknowns. For the secondary fluorescence measurement, a simple calibration curve using fluorescence and turbidity standards may be used. This would most likely require the collection of different transmittance values.

Elimination of Gas Bubble Interference

Bubbles can also drastically affect readings by scattering light. Various embodiments may comprise a back pressure regulator on the sample flow prior to the flow cell 305 to reduce the effect of microbubbles in the sample. Certain embodiments may, either in conjunction with the back pressure regulator or without, comprise a bubble compensation method that may be used for both the primary (λ1) and secondary (λ3) fluorescence measurements. For the secondary fluorescence measurement a Bubble Correction Response Factor (BCRF) may be calculated for a standard solution. This will then be used for ratio calculations for the BCRF of samples. If the ratio is greater than 1, then no action is needed. If the ratio is less than 1, then the turbidity corrected results are multiplied by the ratio. The primary fluorescence measurement will most likely work in a similar fashion. However, unlike the secondary fluorescence measurement method that can be used with turbidity correction, the primary fluorescence measurement method will most likely be non-compatible with turbidity compensation.

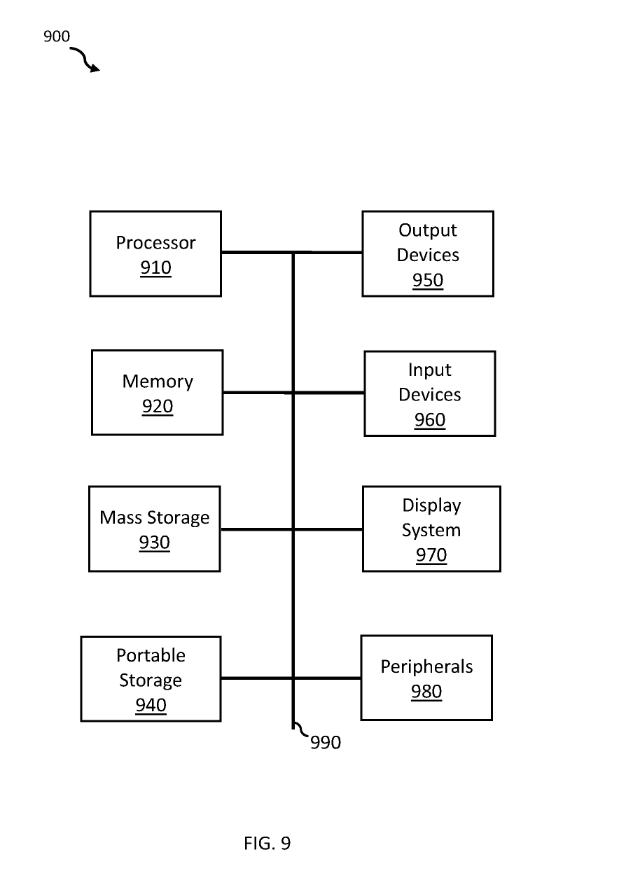

FIG. 9 illustrates an exemplary computing system 900 that may be used to implement an embodiment of the present technology. The computing system 900 of FIG. 9 includes one or more processor units 910 and main memory 920. Main memory 920 stores, in part, instructions and data for execution by processor 910. Main memory 920 can store the executable code when the system 900 is in operation. The system 900 of FIG. 9 may further include a mass storage device 930, portable storage device(s) 940, output devices 950, user input devices 960, a graphics display system 970, and other peripheral devices 980.

The components shown in FIG. 9 are depicted as being connected via a single bus 990. The components may be connected through one or more data transport means. Processor unit 910 and main memory 920 may be connected via a local microprocessor bus, and the mass storage device 930, peripheral device(s) 980, portable storage device(s) 940, and graphics display system 970 may be connected via one or more input/output (I/O) buses.

Mass storage device 930, which may be implemented with a magnetic disk drive or an optical disk drive, is a non-volatile storage device for storing data and instructions for use by processor unit 910. Mass storage device 930 can store the system software for implementing embodiments of the present technology for purposes of loading that software into main memory 920.

Portable storage device 940 operates in conjunction with a portable non-volatile storage media, such as a floppy disk, compact disk or digital video disc, to input and output data and code to and from the computer system 900 of FIG. 9. The system software for implementing embodiments of the present technology may be stored on such a portable media and input to the computer system 900 via the portable storage device 940.

User input devices 960 provide a portion of a user interface. User input devices 960 may include an alphanumeric keypad, such as a keyboard, for inputting alphanumeric and other information, or a pointing device, such as a mouse, a trackball, stylus, or cursor direction keys. Additionally, the system 900 as shown in FIG. 9 includes output devices 950. Suitable output devices include speakers, printers, network interfaces, and monitors.

Graphics display system 970 may include a liquid crystal display (LCD) or other suitable display device. Graphics display system 970 receives textual and graphical information, and processes the information for output to the display device.

Peripheral devices 980 may include any type of computer support device to add additional functionality to the computer system. Peripheral device(s) 980 may include a modem or a router.

The components contained in the computer system 900 of FIG. 9 are those typically found in computer systems that may be suitable for use with embodiments of the present technology and are intended to represent a broad category of such computer components that are well known in the art. Thus, the computer system 900 of FIG. 9 can be a personal computer, hand held computing system, telephone, mobile computing system, workstation, server, minicomputer, mainframe computer, or any other computing system. The computer may also include different bus configurations, networked platforms, multi-processor platforms, etc. Various operating systems can be used including UNIX, Linux, Windows, Macintosh OS, Palm OS, and other suitable operating systems.

Some of the above-described functions may be composed of instructions that are stored on storage media (e.g., computer-readable media). The instructions may be retrieved and executed by the processor. Some examples of storage media are memory devices, tapes, disks, and the like. The instructions are operational when executed by the processor to direct the processor to operate in accord with the technology. Those skilled in the art are familiar with instructions, processor(s), and storage media.

It is noteworthy that any hardware platform suitable for performing the processing described herein is suitable for use with the technology. The terms “computer-readable storage medium” and “computer-readable storage media” as used herein refer to any medium or media that participate in providing instructions to a CPU for execution. Such media can take many forms, including, but not limited to, non-volatile media, volatile media and transmission media. Non-volatile media include, for example, optical or magnetic disks, such as a fixed disk. Volatile media include dynamic memory, such as system RAM. Transmission media include coaxial cables, copper wire and fiber optics, among others, including the wires that comprise one embodiment of a bus. Transmission media can also take the form of acoustic or light waves, such as those generated during radio frequency (RF) and infrared (IR) data communications. Common forms of computer-readable media include, for example, a floppy disk, a flexible disk, a hard disk, magnetic tape, any other magnetic media, a CD-ROM disk, digital video disk (DVD), any other optical media, any other physical media with patterns of marks or holes, a RAM, a PROM, an EPROM, an EEPROM, a FLASHEPROM, any other memory chip or data exchange adapter, a carrier wave, or any other media from which a computer can read.

Various forms of computer-readable media may be involved in carrying one or more sequences of one or more instructions to a CPU for execution. A bus carries the data to system RAM, from which a CPU retrieves and executes the instructions. The instructions received by system RAM can optionally be stored on a fixed disk either before or after execution by a CPU.

Computer program code for carrying out operations for aspects of the present technology may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The corresponding structures, materials, acts, and equivalents of all means or step plus function elements in the claims below are intended to include any structure, material, or act for performing the function in combination with other claimed elements as specifically claimed. The description of the present technology has been presented for purposes of illustration and description, but is not intended to be exhaustive or limiting to the invention in the form disclosed. Many modifications and variations will be apparent to those of ordinary skill in the art without departing from the scope and spirit of the invention. Exemplary embodiments were chosen and described in order to best explain the principles of the present technology and its practical application, and to enable others of ordinary skill in the art to understand the invention for various embodiments with various modifications as are suited to the particular use contemplated.

Aspects of the present technology are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram or blocks.

These computer program instructions may also be stored in a computer readable medium that can direct a computer, other programmable data processing apparatus, or other device to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable data processing apparatus, or other devices to produce a computer implemented process such that the instructions which execute on the computer, other programmable data processing apparatus, or other devices provide processes for implementing the functions/acts specified in the flowchart and/or block diagram block or blocks.

The flowchart and block diagrams illustrate the architecture, functionality, and operation of possible implementations of systems, methods, and computer program products according to various embodiments of the present technology. In this regard, each block in the flowcharts or block diagrams may represent a module, segment, or portion of code, which comprises one or more executable instructions for implementing the specified logical functions(s). It should also be noted that, in some alternative embodiments, the functions noted in the block may occur out of the order noted in the figures. For example, two blocks shown in succession may, in fact, be executed substantially concurrently, or the blocks may sometimes be executed in the reverse order, depending on the functionality involved. It will also be noted that each block of the block diagrams and/or flowchart illustrations, and combinations of blocks in the block diagrams and/or flowchart illustrations, can be implemented by special purpose hardware-based systems that perform the specified functions or acts, or combinations of special purpose hardware and computer instructions.

While various embodiments have been described above, it should be understood that they have been presented by way of example only, and not limitations. The descriptions are not intended to limit the scope of the technology to the particular forms set forth herein. Thus, the breadth and scope of a preferred embodiment should not be limited by any of the above-described exemplary embodiments. It should be understood that the above description is illustrative and not restrictive. To the contrary, the present descriptions are intended to cover such alternatives, modifications, and equivalents as may be included within the spirit and scope of the technology as defined by the appended claims and otherwise appreciated by one of ordinary skill in the art. The scope of the technology should, therefore, be determined not with reference to the above description, but instead should be determined with reference to the appended claims along with their full scope of equivalents.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc. and are also not intended to be limiting. Like terms refer to like elements throughout the description.

As used herein, the terms “having”, “containing”, “including”, “comprising”, and the like are open ended terms that indicate the presence of stated elements or features, but do not preclude additional elements or features. The articles “a”, “an” and “the” are intended to include the plural as well as the singular, unless the context clearly indicates otherwise.

What is claimed is:

1. A method for measuring polycyclic aromatic hydrocarbon concentration of an aqueous stream, comprising:
passing a sample of an aqueous stream through a monitoring area;
directing a first electromagnetic radiation into the monitoring area, the first electromagnetic radiation having a first wavelength;
directing a second electromagnetic radiation into the monitoring area, the second electromagnetic radiation having a wavelength different than the first wavelength;
monitoring at least one fluorescence when the first electromagnetic radiation is directed into the monitoring area;
monitoring at least one fluorescence when the second electromagnetic radiation is directed into the monitoring area; and
determining a polycyclic aromatic hydrocarbon concentration reported as a phenanthrene equivalent concentration using at least one of the monitored fluorescence.

2. The method of claim 1, wherein monitoring at least one fluorescence when the first electromagnetic radiation is directed into the monitoring area comprises monitoring the fluorescence at a wavelength greater than the first wavelength.

3. The method of claim 1, wherein monitoring at least one fluorescence when the second electromagnetic radiation is directed into the monitoring area comprises monitoring the fluorescence at a wavelength greater than the wavelength of the second electromagnetic radiation.

4. The method of claim 1, wherein the wavelength of the first electromagnetic radiation corresponds to a peak of a phenanthrene emission spectrum.

5. The method of claim 4, wherein the wavelength of the first electromagnetic radiation ranges from about 240 nm to about 260 nm.

6. The method of claim 4 wherein the wavelength of the second electromagnetic radiation ranges from about 390 nm to about 410 nm.

7. A device for measuring polycyclic aromatic hydrocarbon concentration of an aqueous stream, comprising:
a monitoring area;
a source of a first electromagnetic radiation, the first electromagnetic radiation having a first wavelength, the source directing the first electromagnetic radiation into the monitoring area;
a source of second electromagnetic radiation, the second electromagnetic radiation having a wavelength different than the first wavelength, the source directing the second electromagnetic radiation into the monitoring area;
at least one detector, the at least one detector detecting at least one fluorescence; and
an analysis module, the analysis module determining a polycyclic aromatic hydrocarbon concentration reported as a phenanthrene equivalent concentration using at least one of the detected fluorescence.

8. The device of claim 7, wherein the wavelength of the second electromagnetic radiation is greater than the wavelength of the first electromagnetic radiation.

9. The device of claim 7, wherein the wavelength of the first electromagnetic radiation corresponds to a peak of a phenanthrene emission spectrum.

10. The device of claim 9, wherein the wavelength of the first electromagnetic radiation ranges from about 240 nm to about 260 nm.

11. The device of claim 9 wherein the wavelength of the second electromagnetic radiation ranges from about 390 nm to about 410 nm.

12. A method for measuring polycyclic aromatic hydrocarbon concentration of an aqueous stream, comprising:
obtaining a sample of the aqueous stream;
directing a first electromagnetic radiation having a first wavelength into the sample;
measuring at a second wavelength a fluorescence of the electromagnetic radiation from the sample, the second wavelength greater than the first wavelength;
directing a second electromagnetic radiation having a third wavelength into the sample, the third wavelength greater than the first wavelength and different than the second wavelength;
measuring at a fourth wavelength a fluorescence of the electromagnetic radiation from the sample, the fourth wavelength greater than the third wavelength; and
determining the polycyclic aromatic hydrocarbon concentration of the sample using the measured fluorescence at the second wavelength and fluorescence at the fourth wavelength, the concentration reported as a phenanthrene equivalent concentration.

13. The method of claim 12, wherein the first and second wavelengths correspond to a peak of a phenanthrene emission spectrum.

14. The method of claim 13, wherein the first wavelength ranges from about 240 nm to about 260 nm.

15. The method of claim 13 wherein the second wavelength ranges from about 350 nm to about 370 nm.

16. The method of claim 12, wherein the fourth wavelength is selected such that the fluorescence measured at the fourth wavelength is a linear function of the polycyclic aromatic hydrocarbon concentration.

17. The method of claim 16, wherein the polycyclic aromatic hydrocarbon concentration is within a range such that the fluorescence measured at the second wavelength is a linear function of the polycyclic aromatic hydrocarbon concentration.

18. The method of claim 16, wherein the fluorescence measured at the fourth wavelength is a linear function of the polycyclic aromatic hydrocarbon concentration up to a concentration of about 1,800 μ/L expressed as phenanthrene equivalent.

19. The method of claim 12, wherein determining the polycyclic aromatic hydrocarbon concentration comprises comparing the fluorescence at the second and fourth wavelengths to one or more calibration curves.

20. The method of claim 19, wherein a first calibration curve relates the fluorescence measured at the second wavelength to the concentration of polycyclic aromatic hydrocarbons expressed as phenathrene equivalent concentration.

21. The method of claim 20, wherein a second calibration curve relates the fluorescence measured at the fourth wavelength to the concentration of polycyclic aromatic hydrocarbons expressed as phenathrene equivalent concentration, and a concentration range of the second calibration curve is greater than a concentration range of the first calibration curve.

22. The method of claim 12, further comprising positioning a sensor to measure fluorescence at the second wavelength such that an angle between the sensor and a source of the first electromagnetic radiation ranges from about 0 degrees to about 180 degrees.

23. The method of claim 22, wherein the angle between the sensor and the source of the first electromagnetic radiation ranges from about 80 degrees to about 110 degrees.

24. The method of claim 12, further comprising positioning a sensor to measure fluorescence at the fourth wavelength such that an angle between the sensor and a source of the second electromagnetic radiation ranges from about 0 degrees to about 180 degrees.

25. The method of claim 24, wherein the angle between the sensor and the source of the second electromagnetic radiation ranges from about 80 degrees to about 110 degrees.

26. The method of claim 12, further comprising generating sequential pulses of electromagnetic radiation at each of the first and third wavelengths.

27. The method of claim 26, further comprising measuring fluorescence at the second wavelength only when pulses of electromagnetic radiation are generated at the first wavelength.

28. The method of claim 26, further comprising fluorescence at the fourth wavelength only when pulses of electromagnetic radiation are generated at the third wavelength.

29. The method of claim 12, wherein the first wavelength is in an ultraviolet portion of the spectrum.

30. The method of claim 12, wherein the third wavelength is in an ultraviolet or visible portion of the spectrum.

31. One or more non-transitory computer-readable media having computer-executable instructions for performing a method by running a software program on a computer, the computer operating under an operating system, the method comprising instructions from the software program for determining measuring polycyclic aromatic hydrocarbon concentration of an aqueous stream, the instructions comprising:
directing electromagnetic radiation having a first wavelength into a sample of an aqueous stream;
measuring at a second wavelength a fluorescence of the electromagnetic radiation from the sample, the second wavelength greater than the first wavelength;
directing electromagnetic radiation having a third wavelength into the sample, the third wavelength greater than the first wavelength and different than the second wavelength;
measuring at a fourth wavelength a fluorescence of the electromagnetic radiation from the sample, the fourth wavelength greater than the third wavelength; and
determining the polycyclic aromatic hydrocarbon concentration of the sample using the measured fluorescence at the second wavelength and fluorescence at the fourth wavelength, the concentration reported as a phenanthrene equivalent concentration.
